(19) United States
(12) Patent Application Publication
(10) Pub. No.: US 2010/0256650 A1
(43) Pub. Date: Oct. 7, 2010

(54) GIPS CUTTER

(76) Inventors: Bang Hyun Kang, Busan (KR); Dong Ho Kang, Busan (KR)

Correspondence Address: PARK LAW FIRM, 3255 WILSHIRE BLVD, SUITE 1110, LOS ANGELES, CA, 90010, US

(21) Appl. No.: 12/671,826

(22) PCT Filed: Jun. 20, 2008
(86) PCT No.: PCT/KR08/03523

§ 371 Date: Feb. 2, 2010

(30) Foreign Application Priority Data
Aug. 3, 2007 (KR) 20-2007-0013051

(51) Int. Cl. A61F 15/02

(52) U.S. Cl. 606/105.5

(57) ABSTRACT

The present invention relates to a cast cutter that has upper and lower cutting blade portions each having opposing cutting blades formed at either side of the upper and lower portions thereof in such a way that the opposing cutting blades of the upper cutting blade portion and the opposing cutting blades of the lower cutting blade portion are operated cooperatively with each other, such that the upper and lower cutting blade portions perform a cutting operation, while forming a cutting range of a predetermined width, thereby minimizing a cutting load generated when cutting a cast to make the cutting operation simplified, and thereby having no limitation on a cutting length to provide an excellent cutting efficiency.

### TECHNICAL FIELD

The present invention relates to a cast cutter having a shape of manually-operated scissors, and more particularly, to a cast cutter that has upper and lower cutting blade portions each having opposing cutting blades formed at either side of the upper and lower portions thereof in such a way that the opposing cutting blades of the upper cutting blade portion and the opposing cutting blades of the lower cutting blade portion are operated cooperatively with each other, such that the upper and lower cutting blade portions perform a cutting operation, while forming a cutting range of a predetermined width, thereby minimizing a cutting load generated when cutting a cast to make the cutting operation simplified, and thereby having no limitation on a cutting length to provide an excellent cutting efficiency.

### BACKGROUND ART

Generally, when a bone in a human body such as an arm, a leg, and so on is broken or fractured, a cast is needed so as to treat the broken or fractured bone.

The cast is applied to the bone region by coating a plaster with a predetermined thickness on a bandage to wrappingly support the broken or fractured bone of the body. As the cast is hardened, it prevents the broken or fractured bone from being moved, such that the broken or fractured bone can be rapidly cured.

When the treatment of the broken or fractured bone is completely finished by the wearing of the cast, the cast should be removed from a wearer.

The removal of the cast is achieved generally by using a cutting instrument like a pair of scissors or a rotary cutter blade.

The cutting instrument like the pair of scissors has some shortcomings in that since the cutting stress caused from a substantially thick cutting surface of a cast is acted against the upper and lower cutting blades thereof, it is difficult to perform a cutting operation and in that when the cast is cut over a predetermined length, the cut portion of the cast interrupts the cutting operation to keep the cutting operation from being continued.

On the other hand, the rotary cutter blade performs the cutting operation in more convenient manner than the cutting instrument like the pair of scissors since the cutter blade is rotatably activated by means of a motor, but if the cutter blade comes into contact with the body inside the cast, a serious safety accident occurs to give fear for a wearer s safety to the wearer upon the removal of the cast and to cause a large amount of dust to be generated from the cast upon the cutting of the cast.

### DISCLOSURE OF INVENTION

### Technical Problem

Accordingly, the present invention has been made in an effort to solve the above problems occurring in the prior arts, and it is an object of the present invention to provide a cast cutter that can be safely used as a pair of scissors, having upper and lower cutting blade portions each having opposing cutting blades formed at either side of the upper and lower portions thereof in such a way that the opposing cutting blades of the upper cutting blade portion and the opposing cutting blades of the lower cutting blade portion are operated cooperatively with each other, such that the upper and lower cutting blade portions perform a cutting operation, while forming a cutting range of a predetermined width, thereby minimizing a cutting load generated when cutting a cast to make the cutting operation simplified, and thereby having no limitation on a cutting length to provide an excellent cutting efficiency.

### Technical Solution

To achieve the above object, there is provided a cast cutter including: upper and lower cutting members; upper and lower handles formed on the rear end portions of the upper and lower cutting members; and upper and lower cutting blade portions formed on the front ends thereof in such a manner as to be rotatably coupled by means of a rotary shaft, wherein the lower cutting blade portion includes a body having a predetermined width and a pair of opposing cutting blades and formed slantly on either side of the body, and wherein the upper cutting blade portion having a pair of opposing cutting blades formed on both sides thereof in such a way as to cooperatively engage with the pair of opposing cutting blades of the lower cutting blade portion, whereby the upper and lower cutting blade portions perform a cutting operation while forming a cutting range of the predetermined width by upwardly and downwardly pressurizing movement.

According to the present invention, the upper cutting member and the upper cutting blade portion are rotatably connected to each other, and the handles formed on the rear end portions of the upper and lower cutting members are rotatably connected to each other at the both ends of a linkage, such that as the linkage is operated when the handles are pressurized, the upper cutting blade portion performs the cutting operation, while being rotated about the rotary shaft.

According to the present invention, further, the upper and lower cutting members have a hydraulic cylinder mounted at the rear end portions thereof, such that as the upper cutting member is moved forwardly and backwardly by the operation of an operating switch, the upper cutting blade portion performs the cutting operation, while being rotated about the rotary shaft.

### Advantageous Effects

According to the present invention, there is provided the cast cutter that can be safely used as a pair of scissors, having upper and lower cutting blade portions each having opposing cutting blades formed at either side of the upper and lower portions thereof in such a way that the opposing cutting blades of the upper cutting blade portion and the opposing cutting blades of the lower cutting blade portion are operated cooperatively with each other, such that the upper and lower cutting blade portions perform a cutting operation, while forming a cutting range of a predetermined width, thereby minimizing a cutting load generated when cutting a cast to make the cutting operation simplified, and thereby having no limitation on a cutting length to provide an excellent cutting efficiency.

### BRIEF DESCRIPTION OF THE DRAWINGS

FIG. 1 is a front view showing a cast cutter according to a first embodiment of the present invention.

FIG. 2 is a side view showing the main parts of the cast cutter according to the first embodiment of the present invention.

FIG. 3 is a front view showing the operating state of the cast cutter of the present invention.

FIG. 4 is a side view showing the main parts of FIG. 3.

FIG. 5 is a front view showing a cast cutter where a hydraulic cylinder is mounted according to a second embodiment of the present invention.

FIG. 6 is a plan view showing the cast cutter of FIG. 5.

### EXPLANATIONS ON THE REFERENCE NUMERALS OF THE MAIN PARTS IN THE DRAWINGS

10: upper cutting member 11,21: handles

12,22: finger grip openings 13,14,23: hinges

15: upper cutting blade portion

17,17′ opposing cutting blades

20: lower cutting member

25: lower cutting blade portion

26: body

27,27′ opposing cutting blades

40: linkage 50: handle part

51: operating switch 60: hydraulic hose

70: hydraulic cylinder 71: piston rod

### BEST MODE FOR CARRYING OUT THE INVENTION

Hereinafter, an explanation on a cast cutter according to the preferred embodiments of the present invention will be given with reference to the attached drawings.

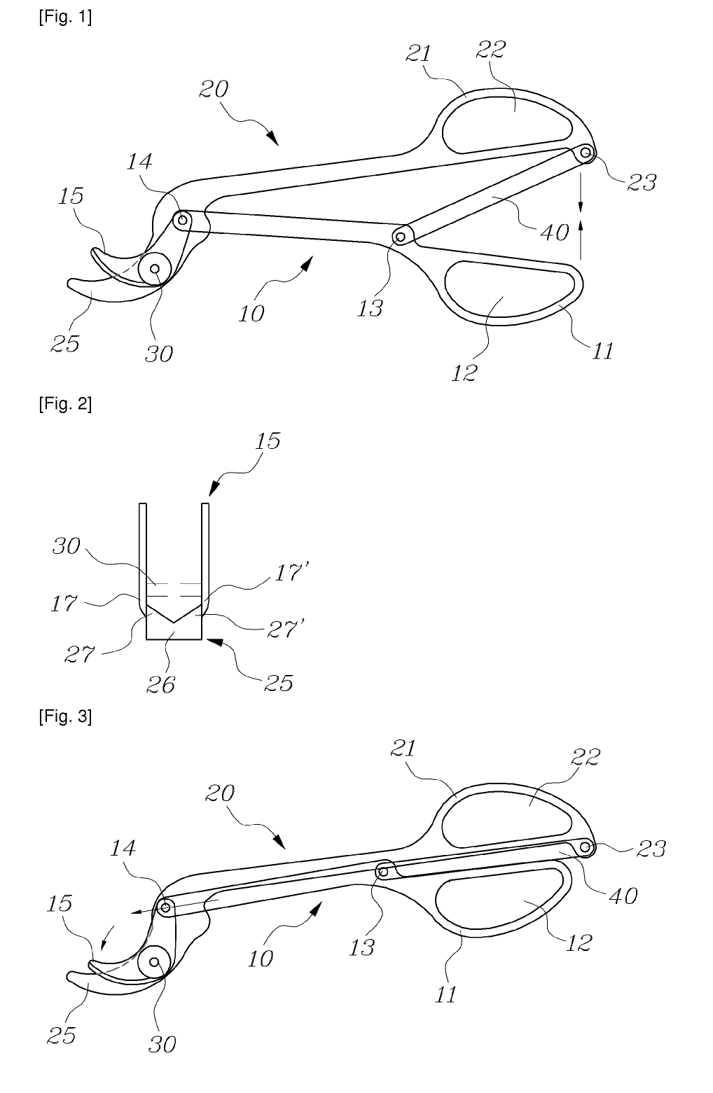

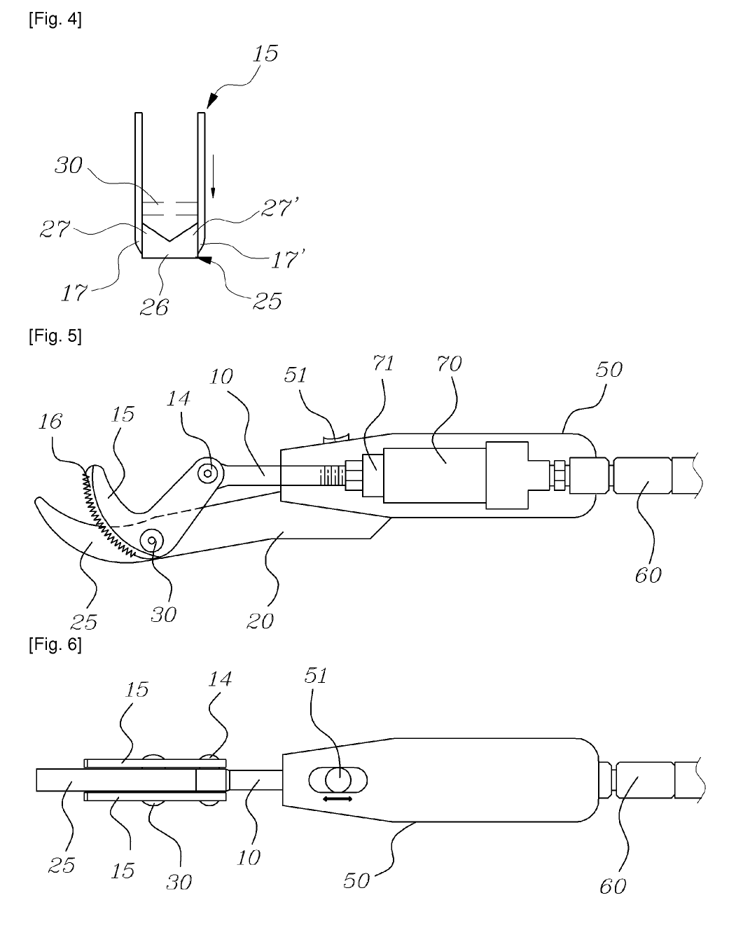

As shown in FIGS. 1 to 4, a cast cutter according to the first embodiment of the present invention comprises upper and lower cutting members 10 and 20, upper and lower handles 11 and 21 formed on the rear end portions of the upper and lower cutting members 10 and 20, and upper and lower cutting blade portions 15 and 25 formed on the front ends thereof in such a manner as to be rotatably coupled by means of a rotary shaft 30. The lower cutting blade portion 25 includes a body 26 having a predetermined width and a pair of opposing cutting blades 27 and 27 formed slantly on either side of the body. The upper cutting blade portion 15 has a pair of opposing cutting blades 17 and 17 formed on both sides thereof in such a way as to cooperatively engage with the pair of opposing cutting blades 27 and 27 of the lower cutting blade portion 25, such that the upper and lower cutting blade portions 15 and 25 perform a cutting operation while forming a cutting range of the predetermined width by upwardly and downwardly pressurizing movement.

In this case, the upper cutting member 10 and the upper cutting blade portion 15 are rotatably connected to each other by means of a hinge 14, and the handles 11 and 21 formed on the rear end portions of the upper and lower cutting members 10 and 20 are rotatably connected to each other by means of the coupling of hinges 13 and 13 at the both ends of a linkage 40, such that as the linkage 40 is operated when the handles 11 and 21 are pressurized, the upper cutting blade portion 15 performs the cutting operation, while being rotated about the rotary shaft 30.

The cutting blades 27 and 27 of the lower cutting blade portion 25 are formed to have a ‘v’-shaped inclined portion toward a center at the inside thereof, while having an inclination angle in a range between 25° and 35°, and the cutting blades 17 and 17 of the upper cutting blade portion 15 have an angle in a range between 40° and 50°, such that cutting load can be minimized.

Reference numerals 12 and 22 which are not explained yet are finger grip openings adapted to insert a user's fingers thereinto so as to hold the handles 11 and 21.

Next, an explanation on the operation and effect of the cast cutter according to the preferred embodiments of the present invention will be given.

According to the present invention, the upper and lower cutting blade portions of the upper and lower cutting members perform the cutting operation, while forming the cutting range of the predetermined width, thereby minimizing the cutting load for the cast and thereby having no limitation on the cutting length to provide an excellent cutting efficiency.

In more detail, after one side of the cast is inserted between the upper and lower cutting blade portions 15 and 25 at the state where the handles 11 and 21 of the upper and lower cutting members 10 and 20 are held by the user s hand, the handles 11 and 21 are pressurized to perform the cutting operation for the cast.

In other words, when the handles 11 and 21 of the upper and lower cutting members 10 and 20 are pressurized, the upper cutting member 10 is moved forwardly by means of the operation of the linkage 40 connected to the handles 11 and 21.

As the upper cutting member 10 is moved forwardly, the upper cutting blade portion 15 hinge-connected to the upper cutting member 10 is pushed at the upper end thereof and is rotated about the rotary shaft 30, thereby performing the cutting operation together with the lower cutting blade portion 25.

At this time, the pair of opposing cutting blades 27 and 27 formed at either side of the body 26 of the lower cutting blade portion 25 in such a manner as to form the predetermined width therebetween are operated cooperatively with the pair of opposing cutting blades 17 and 17 formed at either side of the upper cutting blade portion 15, such that the cast is cut by the cutting range with the predetermined width.

Further, if the hydraulic cylinder 70 is mounted at the rear ends of the upper and lower cutting members 10 and 20, the upper cutting member 10, which is bolt-coupled to the piston rod 71 of the hydraulic cylinder 70, is linearly moved by means of the operation of the hydraulic pump (not shown), and the forwardly and backwardly moving upper cutting member 10 pushes the upper end of the upper cutting blade portion 15 hinge-connected thereto, such that the upper cutting blade portion 15 performs the cutting operation together with the lower cutting blade portion 25, while being rotated about the rotary shaft 30.

Therefore, during the cutting operation through the upper and lower cutting blade portions 15 and 25 of the upper and lower cutting members 10 and 20, since the cast is cut by the cutting range of the predetermined width, the cutting load for the cast against the upper and lower cutting blade portions 15 and 25 becomes decreased, such that the cutting operation is carried out with ease and continuously, thereby completing the cutting for the cast at a time.

### MODE FOR THE INVENTION

Since a substantially large force should be needed for the cutting operation, as shown in FIGS. 5 and 6, a cast cutter may have a hydraulic cylinder mounted thereon, which makes it convenient to use.

A hydraulic cylinder 70 is mounted at the rear ends of the upper and lower cutting members 10 and 20, and a hydraulic pump (which is not shown) is operated by means of an operating switch 51 to allow the hydraulic cylinder 70 to be linearly moved, such that the upper cutting blade portion 15 performs the cutting operation, while being rotated about the rotary shaft 30.

In more detail, the lower cutting member 20 is fixed at a handle part 50 in which the hydraulic cylinder 70 is mounted, and the upper cutting member 10 is bolt-coupled to a piston rod 71 of the hydraulic cylinder 70 in such a manner as to be moved forwardly and backwardly. The forwardly and backwardly moving upper cutting member 10 pushes the front end of the upper cutting blade portion 15 by means of the hinge 14, such that the upper cutting blade portion 15 can be rotated about the rotary shaft 30.

In this case, the operating switch 51 is disposed at the top end of the handle part 50, while being operated in double-acting, single-acting, and stopping ways. In case of the double-acting through the operating switch 51, the cutting operation is continuously performed to cut the cast, and contrarily, the single-acting through the operating switch 51 is needed when the position for cutting is precisely determined or when the cutting operation may be dangerous. The operating switch 51 is embedded in the hydraulic hose 60 in such a manner as to be connected to the hydraulic pump (not shown).

According to a well-known technology, the hydraulic cylinder 70 is configured as a single mechanical part that performs a shock-absorbing function or extends a rod to allow the member connected to the rod to be linearly reciprocated by means of an external pressure or power means like a hydraulic pump in accordance with the structure of a hydraulic circuit.

As shown in FIG. 5, a saw blade 16 is formed on the upper cutting blade portion 15, thereby preventing the upper and lower cutting blade portions 15 and 25 from being slid on the cast.

1. A cast cutter for cutting and removing a cast, comprising:
upper and lower cutting members 10 and 20;
upper and lower handles 11 and 21 formed on the rear end portions of the upper and lower cutting members 10 and 20; and
upper and lower cutting blade portions 15 and 25 formed on the front ends thereof in such a manner as to be rotatably coupled by means of a rotary shaft 30,
wherein the lower cutting blade portion 25 includes a body 26 having a predetermined width and a pair of opposing cutting blades 27 and 27 formed slantly on either side of the body, and
wherein the upper cutting blade portion 15 having a pair of opposing cutting blades 17 and 17 formed on both sides thereof in such a way as to cooperatively engage with the pair of opposing cutting blades 27 and 27 of the lower cutting blade portion 25,
whereby the upper and lower cutting blade portions 15 and 25 perform a cutting operation while forming a cutting range of the predetermined width.

2. The cast cutter according to claim 1, wherein the upper cutting member 10 and the upper cutting blade portion 15 are rotatably connected to each other by means of a hinge 14, and the handles 11 and 21 formed on the rear end portions of the upper and lower cutting members 10 and 20 are rotatably connected to each other at the both ends of a linkage 40 by means of hinges 13 and 23, such that as the linkage 40 is operated when the handles 11 and 21 are pressurized, the upper cutting blade portion 15 performs the cutting operation, while being rotated about the rotary shaft 30.

3. The cast cutter according to claim 1, wherein the upper and lower cutting members 10 and 20 have a hydraulic cylinder 70 mounted at the rear end portions thereof in such a manner as to allow a piston rod 71 of the hydraulic cylinder 70 to be linearly moved by means of the operation of an operating switch 51, such that as the upper cutting member 10 connected to the piston rod 71 is moved forwardly and backwardly, the upper cutting blade portion 15 connected to the upper cutting member 10 by means of the hinge 14 performs the cutting operation, while being rotated about the rotary shaft 30.
